(19) United States
(12) Patent Application Publication
(10) Pub. No.: US 2006/0166285 A1
(43) Pub. Date: Jul. 27, 2006

(54) Charged permeation layers for use on active electronic matrix devices

(76) Inventors: Jainamma Krotz, San Diego, CA (US); David Wong, San Diego, CA (US); Kenny Nguyen, San Diego, CA (US)

Correspondence Address: O'MELVENY & MYERS LLP, 610 NEWPORT CENTER DRIVE, 17TH FLOOR, NEWPORT BEACH, CA, 92660, US

(21) Appl. No.: 11/044,739

(22) Filed: Jan. 26, 2005

(51) Int. Cl. G01N 33/53; C12M 1/34

(52) U.S. Cl. 435/7.5; 435/287.2

(57) ABSTRACT

An electronic device including at least one electrode on a substrate and a permeation layer overlaying the at least one electrode. The permeation layer comprises a polymer having a plurality of negatively-charged moieties. The negatively charged moieties may be carboxylates. In one embodiment, the negatively charged moieties are carboxylates from acrylic acids in the permeation layer. The amount of acrylic acid may be less than about 5 mol %. The permeation layer may also contain streptavidin and/or a surfactant. Methods of using the electronic device are also disclosed in which at least one of the selectively addressable electrodes are biased to at least partially neutralize the negatively charged moieties in the overlying permeation layer. Charged entities can then bind to the permeation layer over the biased electrode.

### FIELD OF THE INVENTION

The present invention provides improved synthetic polymer hydrogel permeation layers for use on active electronic matrix devices for biological assays. The permeation layers, which include charged moieties, have extremely low background resulting from non-specific binding of DNA. In addition, the present invention also provides synthetic polymer hydrogel permeation layers that contain copolymerized attachment sites for nucleic acid probes or other biomolecules.

### BACKGROUND

The following description provides a summary of information relevant to the present invention. It is not an admission that any of the information provided herein is prior art to the presently claimed invention, nor that any of the publications specifically or implicitly referenced are prior art to the invention.

By placing a plurality of nucleic acid probes on a surface, and exposing the surface to a sample containing target nucleic acids, many hybridization reactions may be carried out on a sample at the same time, simultaneously generating hybridization data for several target nucleic acids (the reverse dot-blot technique). Similarly, by immobilizing nucleic acids from several samples onto the surface, several samples may be probed with the same oligonucleotide probe at the same time (the dot-blot technique). Originally, dot-blot and reverse dot-blot hybridizations were carried out using nucleic acid probes crudely blotted onto a nucleic acid-binding membrane or filter. In the past two decades, several tools have been designed to place nucleic acid probes at defined locations in high densities on various types of surfaces (glass, polymers, silicon nitride, etc.) by methods such as physical deposition (e.g., ink-jet, microspray, pin deposition, microchannel deposition) or by in-situ polymerization techniques (e.g., photo-deprotection methods.) Such “microchip” based DNA arrays have been of great interest in recent years due to their. enormous ability to facilitate rapid analysis of genetic information. Although very advanced techniques are utilized to generate these types of arrays, they still employ parallel hybridization of DNA to the immobilized capture probes in a passive mode. In other words, the nucleic acids present in the entire sample volume interact with the entire array surface at the same time, to the same extent.

In contrast, active electronic matrix arrays use an electric field to facilitate the rapid transport and hybridization of DNA on microchips. In general, active matrix array devices contain an array of electronically addressable microelectrodes on a substrate, which provide electric field control over a variety of biomolecular reactions including DNA transport, hybridization, and denaturation. By using the electrodes to apply an electric field to a solution containing charged molecules, such as nucleic acids, the charged molecules can be rapidly transported to and concentrated at the electrodes that are biased opposite the charge of the molecules. This allows the transport of nucleic acid probes or amplicons to the microlocations in a very efficient and specific manner for binding to attachment moieties at the microlocations (a process sometimes referred to as “programming” the locations), allowing the generation of arrays for dot-blot or reverse dot-blot formats. After the probes or amplicons are immobilized at the microlocations, the electric field can again be used to rapidly direct the second hybridization assay component to the microlocation. Thus, electric field regulated hybridization is one to three orders of magnitude faster than passive hybridization under the same conditions, overcoming several of the limitations of passive hybridization.

These arrays, also known as active programmable electronic matrix devices, or APEX devices, have been extensively described, e.g. in U.S. Pat. Nos. 6,051,380 and 6,245,508, incorporated herein by reference in their entirety. In general, the devices comprise an array of individually controllable microelectrodes on a substrate, and optionally comprise additional counter electrodes for opposite biasing. The microelectrodes are overlaid by a thin permeation layer, defining the microlocations of the device above the microelectrodes. In addition to facilitating the attachment of biomolecules by providing a matrix to affix attachment moieties (e.g., streptavidin), the permeation layer separates the biomolecules from the electrode surface where hydrolysis and other potentially detrimental electrochemical reactions can occur. Although the permeation layer retards or prohibits the movement of the biomolecules towards the microelectrode, the permeation layer is sufficiently permeable to small molecules to permit ion exchange between the electrode surface and the buffer medium, allowing an electric current to flow. The active electronic matrix chips usually use electric current and voltage conditions wherein electric current densities are at least 0.04 nA/μm² (about 200 nA for an 80 μm diameter microlocation) and/or potentials sufficient to hydrolyze water. The electric current density is defined as the electric current divided by the area of the electrode used to support it.

Additionally, the effectiveness of the translocation of charged biomolecules such as nucleotide oligomers within an electronically-driven system such as an active electronic matrix chip depends on the generation of the proper gradient of positively and negatively charged electrochemical species by the anode and cathode, respectively. For example, effective nucleic acid (i.e. either DNA or RNA) transport may be accomplished by generation of protons and hydroxyl anions when the potential at the anode is greater than +1.29 V with respect to a ‘saturated calomel electrode’ (SCE). The transport efficiency of charged molecules increases with increasing current density, thus driving the desire for operation at higher voltage drops and current densities and, thus, the need for evermore robust permeation layers.

The application of an electric current through the permeation layer has also been found to produce considerable chemical and mechanical stress on the thin permeation layer coating at the electrode surface. It has been found that when such thin layers are applied onto electrodes without a covalent attachment to the electrode surface, the permeation layer is prone to separate or ‘delaminate’ from the electrode interface. It is believed this delamination is caused by a change in the chemical make-up at the interface between the permeation layer and the electrode resulting from the application of electronic potential at the electrode and by physical disruption from charged ions and gases emanating from the electrode. Thus, the permeation layer must have sufficient mechanical strength and be relatively chemically inert in order to withstand the rigors of changes at the electrode surface without inordinate stretching or decomposition.

Thus, the permeation layer of active electronic matrix devices is an important element in the overall function of the device. It must be sufficiently permeable to small aqueous ions, yet efficiently sequester biomolecules from the electrode surface. In addition, it must be able to withstand significant chemical and mechanical forces while maintaining its integrity and shape. Several materials have been utilized that provide these qualities. Agarose with glyoxal crosslinked streptavidin (SA) has been used as a permeation layer on commercially available, active electronic matrix chips, and the results of electronic hybridization of DNA on these chips has been reported in several publications (e.g., Sosnowski, et al., Proc. Nat. Acad. Sci. USA, 94:1119-1123 (1997), and Radtkey, et al., Nucl. Acids Resrch., 28(7) e17 (2000.))

Agarose is a naturally sourced carbohydrate polymer hydrogel, containing long polymer strands that are crosslinked by non-covalent bonding. Such hydrogels are referred to as “physical hydrogels,” as they derive their structure from non-covalent interactions, as compared to “chemical hydrogels,” which derive their structure from covalent bonds (or cross-links) between the polymer strands. Agarose permeation layers provide good relative fluorescent intensity measurements in nucleic acid assays such as hybridization assays for single nucleotide polymorphisms (SNPs) and short tandem repeat sequences (STRs) in amplicon and capture-sandwich formats, and also in primer-extension type nucleic acid assays that have been used for gene-expression analysis.

Some disadvantages, however, are encountered in the use of agarose as a permeation layer material. Both the manufacturing process and the fact that agarose is a naturally-sourced product introduce some variation, which may vary performance from batch to batch, necessitating increasingly strict quality controls. This is not ideal for large-scale manufacturing. Thus, an alternative material that is not naturally derived, which can be easily formed into a permeation layer on the device, and which will meet or exceed the operating standard of agarose, is greatly desirable.

Polyacrylamide and other synthetic polymer gels offer an alternative to agarose hydrogel permeation layers. These materials are wholly synthetic, and thus offer strict quality control of the components. In addition, they may be easily molded onto the microelectrode array surface with a high degree of uniformity across the entire device. Permeation layers that are between 1 and 2 μm thick in the dry state can be easily produced in this manner, and are amendable to high-throughput manufacture. After molding, streptavidin is covalently linked to the surface of the hydrogel to provide attachment sites for biotinylated oligonucleotide probes or amplicons. Although traditionally formulated polyacrylamide hydrogels made by the micromolding process are uniform, and offer better product control, there still exists a problem with high background noise due to non-specific binding of nucleic acids. Thus, there is still a need for high-performance synthetic polymer hydrogel permeation layers for use on active electronic matrix chip devices.

### SUMMARY OF THE INVENTION

The invention provides a device including at least one electrode on a substrate and a permeation layer overlying the electrode. The permeation layer includes a polymer having a plurality of negatively charged moieties.

The invention also includes an electronic device adapted to receive a solution, which includes a substrate, a plurality of selectively addressable electrodes on the substrate, a permeation layer overlying the electrodes wherein the permeation layer includes a polymer having a plurality of negatively-charged moieties, and an electric source for selectively addressing the electrodes.

The invention also includes a permeation layer comprising a polymer having a plurality of negatively-charged moieties. The polymer can be acrylamide, methylene bis-acrylamide, acrylic acid, and combinations thereof.

The invention also includes a permeation layer comprising a polymer having a plurality of negatively charged moieties and streptavidin.

The invention also includes a method of addressing a charged entity to a selectively addressable electrode including the steps of providing a permeation layer overlying a plurality of selectively addressable electrodes, wherein the permeation layer includes a polymer having a plurality of negatively charged moieties. At least one of the selectively addressable electrodes is biased to at least partially neutralize the negatively-charged moieties. A charged entity is then bound to the permeation layer overlying the at least one selectively addressable electrode. The charged entity may be DNA, RNA, p-RNA, proteins, antibodies, cells, or any other charged moiety.

In all of the above stated embodiments, the plurality of negatively charged moieties may be carboxylates. (COO⁻). More specifically, the negatively charged moieties may be the carboxylates from acrylic acid monomers that were incorporated into the permeation layer. The overall negative charge density of the permeation layer may be less than about or about 5%. The amount of acrylic acid may be less than about 5 mol %. The permeation layer may also include streptavidin. The streptavidin may be modified with acrylamide. Additionally, the permeation layer may also include a surfactant. The surfactant may also be non-ionic, for example, Brij. The amount of surfactant in the permeation layer may be about 2.5 mol % or about 5.0 mol %.

### BRIEF DESCRIPTION OF THE FIGURES

FIG. 1 depicts the influence of negative charge density on pads during electronic activation.

FIG. 2 depicts a modification of streptavidin.

FIG. 3 depicts a polymerization scheme to form a 3-D polymer network.

FIG. 4 depicts theta values of various acrylic acid formulations.

FIG. 5A depicts the amount of binding of oligonucleotides of different lengths to various formulations of permeation layers.

FIG. 5B depicts the amount of binding of oligonucleotides of different lengths to various formulations of permeation layers.

FIG. 6A depicts the results from a SNP assay.

FIG. 6B depicts a comparison of background signals in the SNP assay.

FIG. 7 depicts the amount of binding of oligonucleotides with varying concentrations of acrylic acid and surfactant.

FIG. 8A depicts the amount of binding of oligonucleotides with varying concentrations of acrylic acid and surfactant.

FIG. 8B depicts the amount of binding of oligonucleotides with varying concentrations of acrylic acid and surfactant.

FIG. 9 depicts the amount of binding of oligonucleotides with varying concentrations of acrylic acid and surfactant.

FIG. 10 depicts the amount of binding of oligonucleotides with varying concentrations of acrylic acid and surfactant.

FIG. 11A depicts the amount of binding of oligonucleotides with varying concentrations of acrylic acid and surfactant.

FIG. 11B depicts the amount of binding of oligonucleotides with varying concentrations of acrylic acid and surfactant.

FIG. 12 depicts the amount of binding of oligonucleotides with varying concentrations of acrylic acid and surfactant.

FIG. 13 depicts the amount of background signal in the ApoE SNP assay.

### DETAILED DESCRIPTION OF THE INVENTION

As has been described, the permeation layer, which overlies the electrodes of the microlocations or active sites, plays a key role in the function of active electronic matrix devices. As part of its function, the permeation layer provides attachment moieties for the attachment and immobilization of nucleic acids (or other specific binding entities, such as antibodies, or synthetic binding moieties such as pyranosyl-RNA or pyranosyl-DNA). More importantly, the permeation layer separates the attached or tethered oligonucleotides and hybridized target DNA sequences from the highly reactive electrochemical environment generated immediately at the electrode surface. This highly reactive electrode surface, and the electrochemical products concentrated at the electrode surface, can rapidly destroy DNA probes and target DNA sequences that contact the surface or approach it too closely. Similar detrimental effects may be encountered with other macromolecular binding entities immobilized directly on the electrode surface. The permeation layer allows oligonucleotides and DNA fragments to be electronically concentrated above, rather than on, the electrode surface and hybridized to anchored complementary oligonucleotides while being protected from the reactive electrode surface and its immediate environment. The permeation layer also allows the gradual diffusion of the electrochemical reaction products (H⁺, OH⁻, gasses, etc.) into the solution around the microlocation, allowing these products to balance the charge through the permeation layer by ion exchange and to react with buffer species. Thus, the design of the microelectrode and permeation layer, forming a microlocation structure, allows high current densities to be achieved in a very confined area, while minimizing the adverse effects produced by the electrode itself.

Once specific binding entities, such as nucleic acids, have been addressed to microlocations and immobilized, the addressed devices are able to control and actively carry out a variety of assays and reactions. Analytes or reactants can be transported by free field electrophoresis to any specific microlocation where the analytes or reactants are effectively concentrated and reacted with the specific binding entity at the microlocation. The sensitivity for detecting a specific analyte or reactant in dilute sample solutions is improved because of this concentrating effect. An additional advantage, which also improves the specificity of the assays carried out on the device, is that any unbound analytes or reactants can be removed by reversing the polarity of a microlocation (also known as “electronic washing”).

The ability to produce a precisely controlled high current level, or density, at individual microlocations even allows the selective “de-hybridization” of DNA fragments, achieving hybridization selectivity at the level of single base mismatches. Thus, the devices can further improve the specificity of assays and reactions by providing another parameter to encourage mismatch de-hybridization (along with the more traditional parameters of temperature and chemical environment), which is known as “electronic stringency,” or “electronic stringency control (ESC).” For DNA hybridization reactions that require different stringency conditions, ESC overcomes an inherent limitation of conventional array technologies, which must rely on stringency conditions that are consistent for all sites over the entire array. The active devices of this invention can electronically produce different stringency conditions at each microlocation. This adds another controllable factor affecting hybridization, along with the more traditional factors such as temperature, salt concentration, and the presence of chaotropic agents. Thus, all hybridizations can be carried out optimally in the same bulk solution, and multiple hybridization reactions can be carried out with minimal outside physical manipulations. Additionally, it may be unnecessary to change temperature in some cases, and the need for multiple washing procedures is greatly reduced.

Thus, the permeation layer of active electronic matrix devices is more than simply a mechanical support to hold attachment sites for specific binding entities. It is also an important factor in the overall performance and efficiency of the devices in their active electronic modes. Unlike coatings or gel supports that have been described for passive array devices, e.g., the gel-block arrays described in U.S. Pat. No. 5,770,721, which simply use hydrogel matrices as an attachment scaffold, permeation layers used on the active electronic matrix devices described herein must also allow the efficient active electronic transport of biomolecules to the microlocations of the device, and be conducive to electronic hybridization and/or stringency procedures.

The two main requirements for permeation layers are (1) high density of attachment of the DNA (capture probes, sample, etc.) to enable a high level of sensitivity of detection and (2) very low background resulting from the low nonspecific binding of DNA. Due to the high affinity of streptavidin and biotin, incorporation of streptavidin in a polymer membrane provides a matrix for the binding of biotinylated DNA. But as a result of steric interactions and other inherent limitations of a polymer immobilized protein, achieving a high level of DNA attachment in such a matrix is a challenge. A porous hydrogel matrix that provided a robust platform for electronic DNA attachment and single nucleotide polymorphism (SNP) detection was previously described in U.S. application Ser. No. 10/014,895, filed on Dec. 10, 2001, which is herein expressly incorporated by reference in its entirety. The hydrogel was design by incorporating streptavidin in a polyacrylamide matrix. The hydrogel was engineered to be porous by a “surfactant templating” approach. Robust and reproducible assay performance on this standard hydrogel for a number of assays in which a sample amplicon is coupled to the permeation layer (including SNP's assays) have been established. The present invention describes a permeation layer, and method of making and use of said permeation layer, that minimizes non-specific binding of DNA under electronic addressing conditions onto DNA arrays.

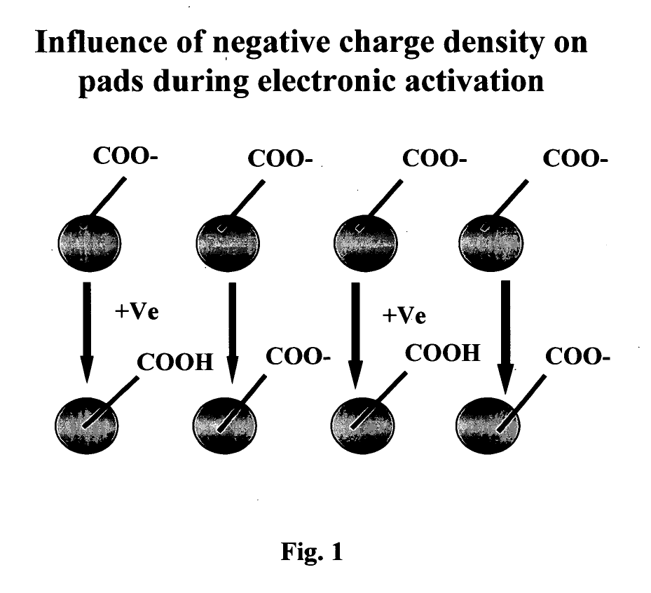

Incorporation of negatively charged moieties into the hydrogel has been found to lower the background signal resulting from non-specific binding of charged biomolecules. The negative charge density of the permeation layer minimizes the nonspecific binding of any negatively charged biomolecules via charge repulsion. Negatively charged biomolecules include but are not limited to DNA, RNA, p-RNA, proteins, cells, or any other charged moiety. As seen in FIG. 1, during positive biasing of the microelectrodes at a specific microlocation (or pad), the protons generated at the electrode neutralize the charge density over the microlocation. Therefore, the microlocations that are positively biased do not have a net charge and therefore, offer a favorable matrix for binding negatively charged biomolecules, e.g., biotinylated DNA. Thus, the DNA can concentrate and subsequently attach or couple to the hydrogel via the streptavidin-biotin interaction. In contrast, the unaddressed microlocations still have a net negative charge density and therefore, serves to repel the negatively charged DNA molecules, thereby minimizing nonspecific DNA binding by charge repulsion.

Additionally, the swelling of the hydrogel is also affected by the incorporation of charged entities into the permeation layer. The water hydration is increased in these permeation layers through charge interaction. The negatively charged biomolecules are thought to repel each other, thereby increasing the overall porosity of the permeation layer. This results in a matrix that is more porous in the wet state when compared to its non-ionic counterpart.

The negatively charged permeation layer can be created by copolymerizing a negatively charged monomer with other monomers. The negatively charged monomer includes, but is not limited to, acrylic acid, methacrylic acid, trichloroacrylic acid, 4-vinylbenzoic acid, 2-acrylamido-2-methyl propanesulfonic acid, and (acryloylamino) (hydroxyl) acetic acid, and combinations thereof. The other monomers are preferably hydrophilic. These include but are not limited to acrylamide, methylene bisacrylamide, acrylic modified streptavidin, and combinations thereof. In one embodiment, acrylic acid is used as the negatively charged monomer. The acid group of acrylic acid has a pKa of approximately 4. Therefore, the acid group of acrylic acid gets protonated during the positive biasing of the electrode.

The method of synthesizing the charged permeation layer allows for the control and adjustment of the degree of incorporation of the charged monomer. The estimated charge density of the permeation layer, based on the quantified molar feed ratio, is preferably less than about or about 10%, alternatively less than about or about 9%, alternatively less than about or about 8%, alternatively less than about or about 7%, alternatively less than about or about 6%, alternatively less about than or about 5%, alternatively less than about or about 4%, alternatively less about than or about 3%, alternatively less than about or about 2%, alternatively less than about or about 1%. In one embodiment, the negative charge density is preferably less than about or about 5%. When the charge density is above this range, the concentration and subsequent coupling of DNA is the pads is low.

The permeation layer of this invention can serve as a universal platform for electronic DNA arrays serving a wide range of applications using DNAs of various lengths. Previously, the hydrogels with different theta values had to be used depending on the length of DNA to be coupled to the permeation layer. For assays using long DNA molecules (greater than 70 bases), hydrogels with theta values of approximately 3.0-3.8 were used. In contrast, for assays using shorter DNA molecules (less than 70 bases), hydrogels with theta values of approximately 2.0-3.0 were used. The permeation layers containing the negatively charged moieties with theta values of approximately 2-4, alternatively 2-3.5, alternatively 2.25-3.5, alternatively 2.5-3.5, alternatively 3.0-3.5, alternatively about 3.3, are able to serve as a good matrix for both short (less than 70 bases) and long (greater than 70 bases) DNA molecules.

### EXAMPLE 1

### Preparation of Permeation Layer

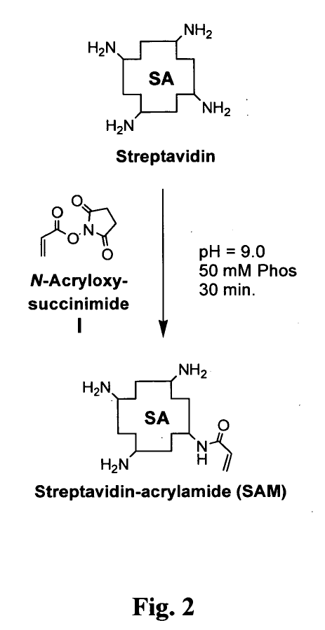

Streptavidin was modified as outlined in FIG. 2. The amine of streptavidin was coupled with N-acryloxysuccinimde. The modified streptavidin (SAM) was purified by size exclusion chromatography using a G-50 Sephadex column followed by concentration via ultrafiltration (Amicon).

Platinum (Pt) substrates having 10×10 electronically addressable array pads were subjected to plasma cleaning (Ar, 15 min) and subsequently silanized with Bind Silane using vapor deposition silanization technique (See U.S. application Ser. No. 09/464,670, issued as U.S. Pat. No. 6,303,087, which is hereby incorporated by reference in its entirety). This silanization procedure introduced polymerizable acrylic groups on the Pt surface that can then be copolymerized with the monomers.

The monomer solution was prepared by dissolving 2.42 g of acrylamide and 0.58 g methylene bisacrylamide (Bis) in 7.0 ml water. A known weight of a surfactant (e.g., Brij 700) was dissolved in the monomer solution at room temperature. The mixture was vortexed to ensure complete dissolution. Hydrogel formulations with higher crosslink density were prepared with greater than 10 mol percent of Bis. An example of such formulation is a formulation where the ratio of acrylamide:Bis is 85:15 mol/mol.

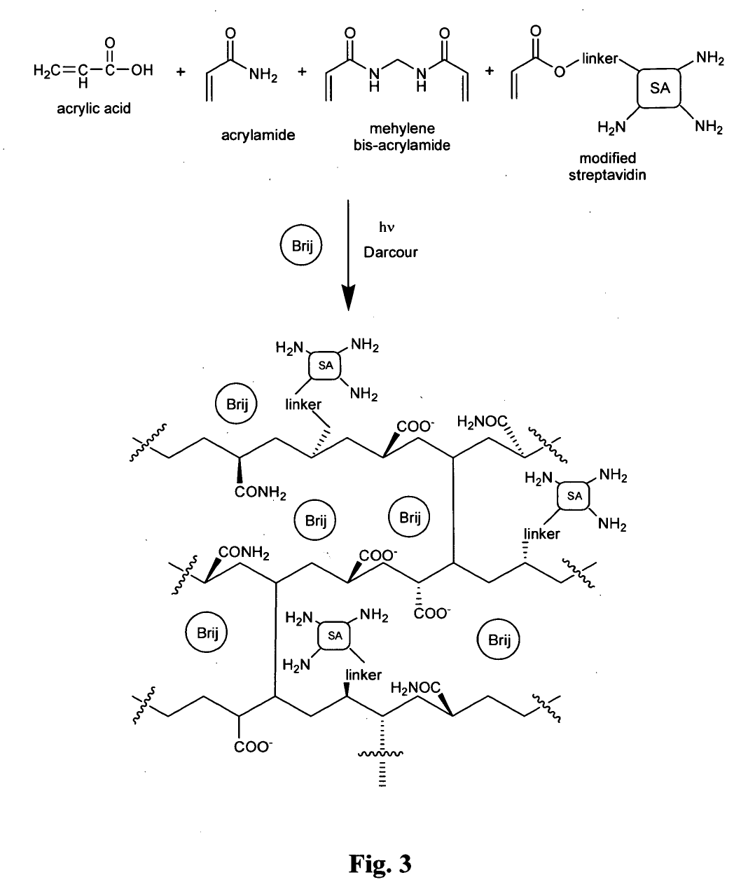

As seen in FIG. 3, the modified streptavidin is copolymerized with hydrophilic monomers such as acrylamide, methylene bis actylamide, and acrylic acid using Darcour 4265 as the initiator. Monomer solutions with various amounts of acrylic acid were prepared (see Table 1). Formulations were prepared with various amounts of a nonionic surfactant, such as Brij 700 (see Table 2). This nonionic surfactant functions as a porogen. The charged monomer (e.g., acrylic acid) content varied from about 0.5 to 3.5 mol %, while the surfactant content (e.g., Brij) varied from about 0 to 5%. As stated previously, it is believed that less surfactant can be used in these formulations containing a charged monomer because the charge repulsion between the charged monomers are thought to create additional pores.

TABLE 1
Monomer Solutions with Acrylic Acid

| Formulation | Monomer Solution | Acrylic Acid | |
|---|---|---|---|
| No. | (ml) | (μl) | % Acrylic Acid |
| 1 | 2.5 | 5 | 0.7 |
| 2 | 2.5 | 10 | 1.4 |
| 3 | 2.5 | 15 | 2.2 |
| 4 | 2.5 | 20 | 2.9 |

TABLE 2

| | Monomer solution with | |
|---|---|---|
| | acrylic acid | |
| Formulation No. | (formulation no. from Table 1) | Brij (w/v %) |
| 5 | Formulation 1 (300 μl) | 0 |
| 6 | Formulation 2 (300 μl) | 0 |
| 7 | Formulation 3 (300 μl) | 0 |
| 8 | Formulation 4 (300 μl) | 0 |
| 9 | Formulation 1 (300 μl) | 2.5 |
| 10 | Formulation 2 (300 μl) | 2.5 |
| 11 | Formulation 3 (300 μl) | 2.5 |
| 12 | Formulation 4 (300 μl) | 2.5 |
| 13 | Formulation 1 (300 μl) | 5.0 |
| 14 | Formulation 2 (300 μl) | 5.0 |
| 15 | Formulation 3 (300 μl) | 5.0 |
| 16 | Formulation 4 (300 μl) | 5.0 |

In the absence of surfactants, the polymerization proceeds in a continuous phase resulting in homogeneous, non-phase separated polymer gels. In the presence of a porogen such as a surfactant assumes an ordered structure (e.g., hexagonal, bicontinuous cubic, or lamellar) and the monomers are dissolved in an aqueous phase surrounding this ordered phase. The polymer chains are formed around the surfactant assemblies in order to stabilize the ordered structure. Subsequent removal of the surfactant from the three-dimensional polymer network leaves behind voids that act as pores.

Characterization of the Hydrogel Permeation Layers

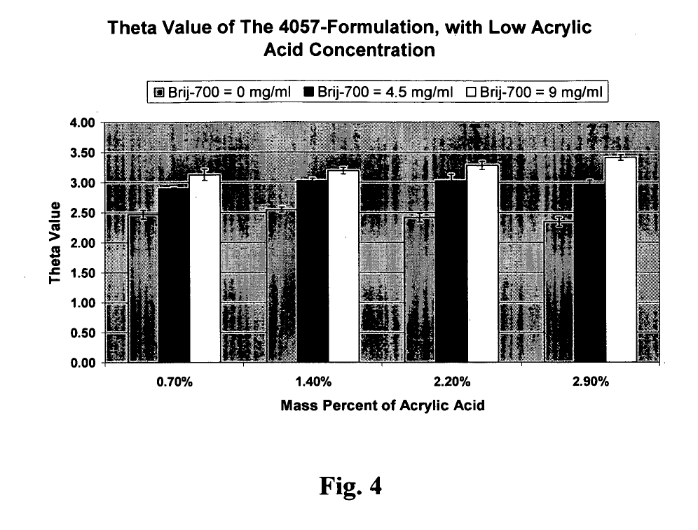

The morphology of the various hydrogel formulations has been analyzed by confocal microscopy and scanning electron microscopy (SEM). Using confocal microscopy, the amount of light scattered from a dry hydrogel was quantified and expressed as a “theta” value (see discussion below), which is a relative measurement of light scattering compared to a non-scattering surface. SEM analyzes the swollen hydrogels after critical point drying. Theta values of a series of a series of formulations with low acrylic acid are shown in FIG. 4.

As discussed in U.S. application Ser. No. 10/014,895, which is hereby incorporated by reference in its entirety, a dimensionless parameter, θ (theta), was used to express the degree of phase separation, or porosity, based on light scattering measurements under the dark field microscope. The dimensionless degree of phase separation (θ) was determined by integrating the dark filed light intensity readings a dry hydrogel layer on the test chip (λ), a standard layer (λₛ) with a medium degree of phase separation and a non-phase separated, or solid, layer (λ₀) on the Leica INM 100 dark field microscope, and was computed with the following formula. When λ₀<<1 the equation can be simplified:

$$θ ≡ \frac{λ - {λ}_{0}}{{λ}_{S} - {λ}_{0}} \approx \frac{λ}{{λ}_{S}}$$

An example of a surface that would approach an ideal non-phase separated layer would be a very smooth surface, such as vapor-deposited platinum on an electronics grade silicon wafer.
Performance of Hydrogels in Assays

A number of different DNA binding and hybridization assays were used to evaluate the performance of these hydrogels as platforms for electronic DNA arrays.

DNA oligonucleotides of various lengths were tested in a series of synthetic capture binding and hybridization assays to compare their extent of binding to the various formulations and to determine the effect of increasing negative charge on DNA binding.

In the electronic capture loading and hybridization assays, the cartridge was equilibrated with 50 mM Histidine for 30 minutes at room temperature. Subsequently, the cartridge was washed three times with 50 mM Histidine and then loaded into molecular biology workstation (MBW) loader. A solution of the biotinylated capture probes were prepared in a 50 mM Histidine solution at a concentration of 10 nM. A solution of the reporter probes was prepared in a 50 mM Histidine solution at a concentration of 200 nM. The capture probes and reporter probes were transferred to a 96-well plate and in the loader and electronic loading was performed under the following conditions:
Capture Address:
   Addressing Voltage=2.0 v
   Addressing Duration=1.0 minute
   Number of Pads Biased Per Address=10
   (Address 50 mM Histidine to a set of 10 pads as background)
Hybridization:
   Addressing Voltage=2.0 v
   Addressing Duration=2.0 minutes
   Number of Pads Biased Per Address=10
      After the loading, the cartridges were read in the MBW-reader by measuring the fluorescence intensity of the pads. The capture oligonucleotides used were T12 (a 5′-biotinylated 12-mer with a CY3 label at the 3′ end), ATA5 (a 5′-biotinylated 19-mer without any fluorophores labels), and 3133 (a 5′-biotinylated 46-mer with a CY3 fluorophore at the 3′ end). The reporter probes RCA5 and 3100 that were used were complementary sequences to ATA5 and 3133, respectively, and were labeled with CY5 fluorophore.

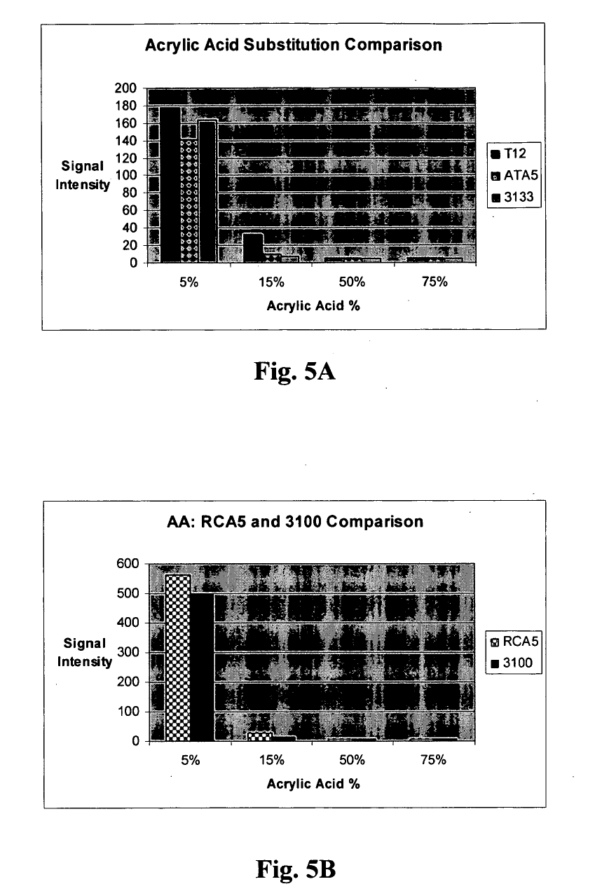

FIGS. 5A and 5B illustrate the results of assays in which capture probes of differing lengths (12 nucleotides, 19 nucleotides, and 46 nucleotides) were coupled to permeation layers containing various amounts of acrylic acid. As stated above, the capture probes were biotinylated at the 5′ end and coupled to a fluorophores (Cy3) at the 3′ end. In this assay, the degree of direct binding of the capture probe and the subsequent binding of a reporter labeled with Cy5 is determined. The results indicate that the degree of negative charge density in the hydrogel was found to have a significant impact on DNA binding. At high negative charge density (greater than 5 mol % of acrylic acid), there is only minimal binding of the probes to the hydrogel. It is believed that the amount of protons generated under positive biasing of the electrodes was not sufficient to neutralize the negative charge density of the high acrylic acid formulations. Therefore, the negative charge density was not neutralized and the amount of DNA binding was significantly reduced.

In a single nucleotide polymorphism (SNP) assay, the absolute signal intensity for each allele was measured, as well as the discrimination between the two alleles. The cartridge was equilibrated with 50 mM Histidine for 30 minutes at room emperature. The cartridge was subsequently washed three times with 50 mM Histidine and loaded into the MBW loader. A solution of the biotinylated PCR-amplified (desalted) double-stranded DNA was prepared in 50 mM Histidine at a concentration of 5 nM. EH1 amplicons (approximately 120 nucleotides in length) were electronically addressed onto several pads. ApoE amplicons (approximately 225 nucleotides in length) were used as a control. The addressing conditions were as follows:
Addressing Voltage=2.0 v
Addressing Duration=2.0 minutes
Number of Pads Biased Per Address=10
(Address 50 mM Histidine to a set of 10 pads as background) Following the amplicon and Histidine address, the cartridge was incubated with 0.3 N NaOH for 3 minutes in order to denature the double-stranded DNA. The cartridge was then washed five-times with 10 mM Histidine.

Passive hybridization was then performed as follows. The reporter probes were diluted in a high salt buffer (50 mM sodium phosphate/500 mM sodium chloride) to a concentration 500 nM. The cartridge was washed three times with the high salt buffer and then incubated for 3 minutes at room temperature with a reporter probe solution. The cartridge was then washed with a low salt buffer and thermal stringency on the MBW reader was performed. The Cy3 labeled reporter hybridized specifically to the c-allele and the Cy5 labeled reporter hybridized to the t-allele.

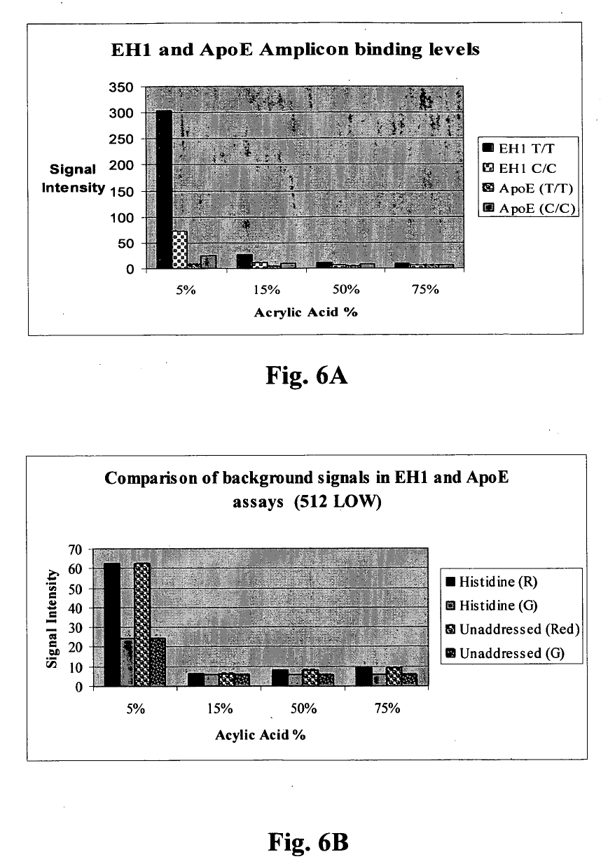

FIGS. 6A and 6B illustrate the results of these SNP assays. At high acrylic acid content, DNA binding is adversely affected by the high charge density. There is also no dependence on length above a specific percentage of acrylic acid, as seen in comparing EH1 and ApoE.

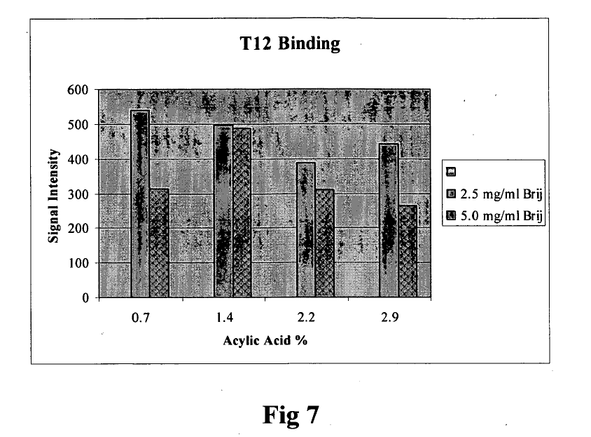

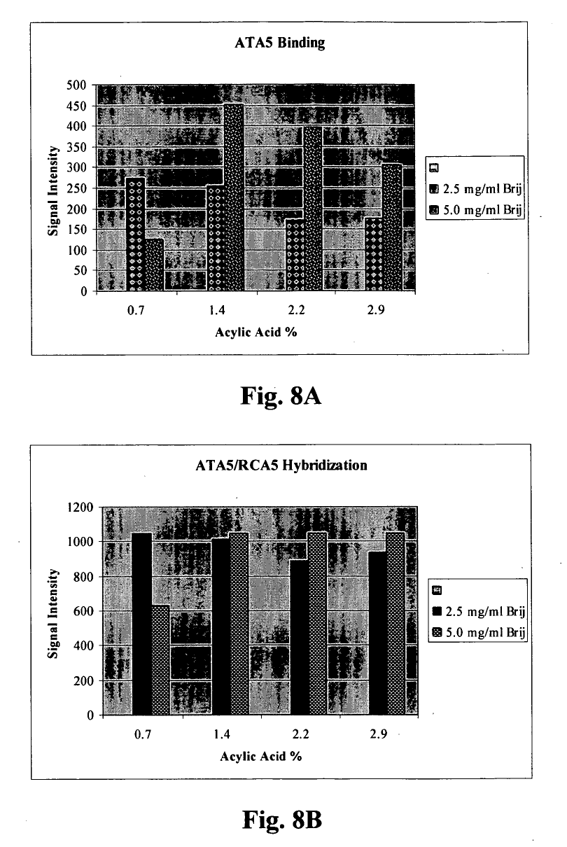

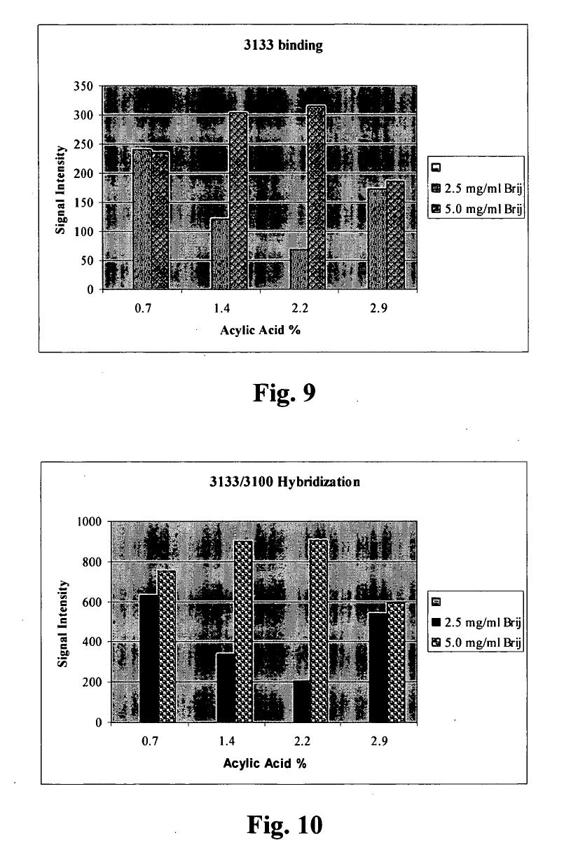

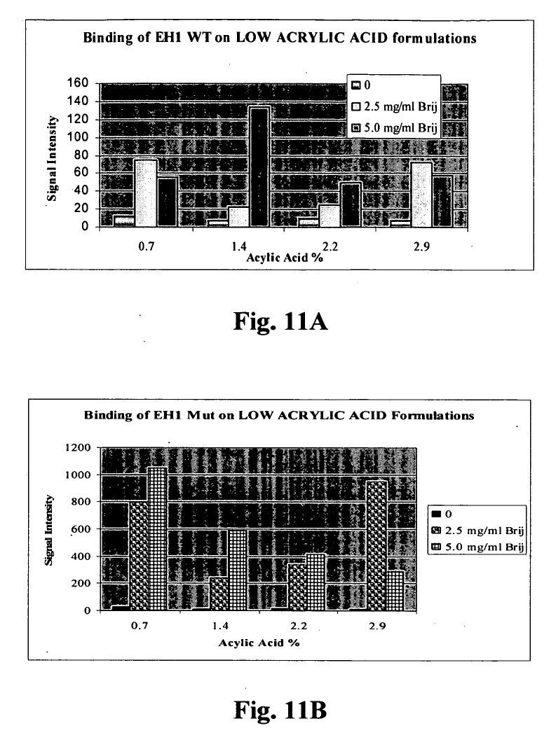

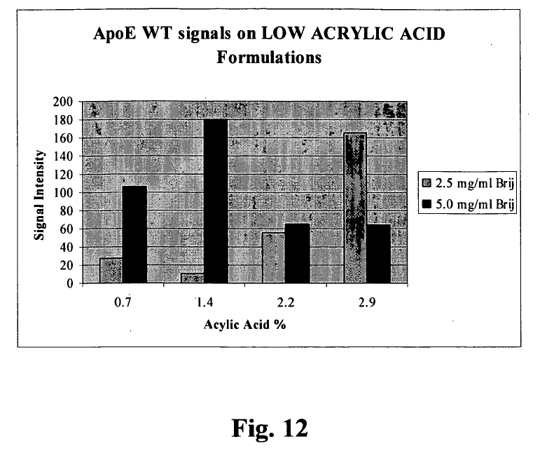

FIGS. 7-12 illustrate the results of different surfactant concentrations and the corresponding effect on signal intensity. FIG. 7 illustrates the binding of a DNA oligonucleotide that is 12 nucleotides long on permeation layers with varying acrylic acid concentrations and varying amounts of surfactant. FIGS. 8A and 8B illustrate the binding of a DNA oligonucleotide that is 19 nucleotides long on permeation layers with varying acrylic acid concentrations and varying amounts of surfactant. FIG. 9 illustrates the binding of a DNA oligonucleotide that is 46 nucleotides long on permeation layers with varying acrylic acid concentrations and varying amounts of surfactant. FIG. 10 illustrates the degree of binding of reporter probe 3100, which is complementary to bound 3133, on permeation layers with varying acrylic acid concentrations and varying amounts of surfactant. FIGS. 11A and 11B illustrate the binding of a DNA oligonucleotide that is approximately 120 nucleotides long on permeation layers with varying acrylic acid concentrations and varying amounts of surfactant. FIG. 12 illustrates the binding of a DNA oligonucleotide that is approximately 225 nucleotides long on permeation layers with varying acrylic acid concentrations and varying amounts of surfactant.

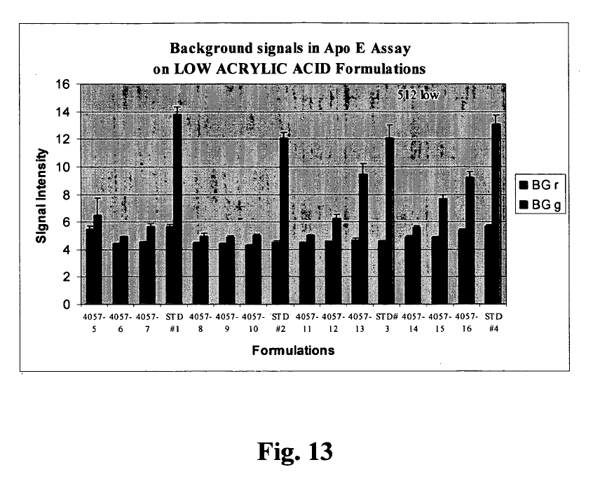

The formulations also exhibit very low background signal from nonspecific DNA binding. FIG. 13 illustrates the background signals in ApoE SNP assay on comparison to standard hydrogels (denoted by STD #1, STD #2, STD #3, and STD #4). In comparison to the standard hydrogels, the formulations containing the negatively charged monomers have significantly lower levels of nonspecific binding.

The formulations containing the charged moiety also show a reduced level of “capture carry over.” As seen in Table 3, which contains raw data for a standard hydrogel, when a capture probe is addressed to the microlocations located in the first row, some amount of carry over can be seen to the adjacent rows (relatively higher signal levels in row 2). In contrast, as seen in Table 4, which contains raw data for a permeation layer containing acrylic acid, both the background signal and amount of carry over has been reduced.

TABLE 3
Raw Data for Standard Hydrogel

| Row/ | | | | | | | | | | |
|---|---|---|---|---|---|---|---|---|---|---|
| Col | 1 | 2 | 3 | 4 | 5 | 6 | 7 | 8 | 9 | 10 |
| 1 | 523 | 467 | 441 | 438 | 432 | 410 | 398 | 385 | 352 | 401 |
| 2 | 31 | 31 | 30 | 30 | 27 | 27 | 28 | 27 | 26 | 26 |
| 3 | 23 | 23 | 21 | 20 | 23 | 21 | 21 | 20 | 20 | 19 |
| 4 | 22 | 22 | 21 | 19 | 21 | 18 | 19 | 20 | 19 | 19 |
| 5 | 22 | 22 | 20 | 18 | 21 | 20 | 19 | 20 | 19 | 20 |
| 6 | 22 | 22 | 20 | 19 | 20 | 20 | 19 | 20 | 20 | 19 |
| 7 | 20 | 20 | 20 | 17 | 21 | 21 | 21 | 20 | 19 | 20 |
| 8 | 21 | 21 | 22 | 22 | 21 | 20 | 20 | 20 | 20 | 19 |
| 9 | 19. | 19 | 22 | 21 | 20 | 20 | 20 | 20 | 20 | 19 |
| 10 | 22 | 22 | 22 | 21 | 21 | 21 | 21 | 19 | 20 | 19 |

TABLE 4
Raw Data for Charged Hydrogel

| Row/ | | | | | | | | | | |
|---|---|---|---|---|---|---|---|---|---|---|
| Col | 1 | 2 | 3 | 4 | 5 | 6 | 7 | 8 | 9 | 10 |
| 1 | 412 | 407 | 400 | 403 | 357 | 364 | 371 | 372 | 313 | 309 |
| 2 | 14 | 17 | 15 | 13 | 11 | 9 | 10 | 9 | 11 | 10 |
| 3 | 6 | 6 | 6 | 6 | 6 | 6 | 6 | 6 | 6 | 6 |
| 4 | 5 | 5 | 5 | 5 | 5 | 5 | 5 | 6 | 5 | 5 |
| 5 | 5 | 5 | 5 | 5 | 5 | 5 | 5 | 5 | 5 | 5 |
| 6 | 5 | 5 | 5 | 5 | 5 | 5 | 5 | 5 | 5 | 5 |
| 7 | 5 | 5 | 5 | 5 | 5 | 5 | 5 | 5 | 5 | 5 |
| 8 | 5 | 5 | 5 | 5 | 5 | 5 | 5 | 5 | 5 | 5 |
| 9 | 5 | 5 | 5 | 5 | 5 | 5 | 5 | 5 | 5 | 5 |
| 10 | 5 | 5 | 5 | 5 | 5 | 5 | 5 | 5 | 5 | 5 |

Although the foregoing invention has, for the purposes of clarity and understanding, been described in some detail by way of illustration and example, it will be obvious that certain changes and modifications may be practiced which will still fall within the scope of the appended claims. It will also be understood that any feature or features from any one embodiment, or any reference cited herein, may be used with any combination of features from any other embodiment.

1. A device comprising:
at least one electrode on a substrate; and
a permeation layer overlying the at least one electrode, wherein the permeation layer comprises a polymer having a plurality of negatively-charged moieties.

2. The device of claim 1, wherein the negatively charged moieties are carboxylates.

3. The device of claim 1, wherein the negatively charged moieties comprise acrylic acid.

4. The device of claim 3, wherein the amount of acrylic acid is less than about 5 mol %.

5. The device of claim 1, further comprising streptavidin.

6. The device of claim 5, wherein the streptavidin is modified with N-acryloxysuccinimde.

7. The device of claim 1, further comprising a surfactant.

8. The device of claim 7, wherein the surfactant is non-ionic.

9. The device of claim 7, wherein the surfactant is Brij.

10. The device of claim 7, wherein the amount of surfactant is about 2.5 mol %.

11. The device of claim 7, wherein the amount of surfactant is about 5.0 mol %.

12. An electronic device adapted to receive a solution comprising:
a substrate;
a plurality of selectively addressable electrodes on the substrate;
a permeation layer overlying the electrodes, the permeation layer comprising a polymer having a plurality of negatively-charged moieties; and
an electric source for selectively addressing the electrodes.

13. The electronic device of claim 12, wherein the negatively charged moieties are carboxylates.

14. The electronic device of claim 12, wherein the negatively charged moieties comprise acrylic acid.

15. The electronic device of claim 14, wherein the amount of acrylic acid is less than about 5 mol %.

16. The electronic device of claim 14, further comprising streptavidin.

17. The electronic device of claim 16, wherein the streptavidin is modified with N-acryloxysuccinimde.

18. The electronic device of claim 12, further comprising a surfactant.

19. The electronic device of claim 18, wherein the surfactant is non-ionic.

20. The electronic device of claim 18, wherein the surfactant is Brij.

21. The electronic device of claim 18, wherein the amount of surfactant is about 2.5 mol %.

22. The electronic device of claim 18, wherein the amount of surfactant is about 5.0 mol %.

23-55. (canceled)
